(19) United States
(12) Patent Application Publication
(10) Pub. No.: US 2017/0041586 A1
(43) Pub. Date: Feb. 9, 2017

(54) DEVICE AND METHOD FOR FORMAT CONVERSION OF FILES FOR THREE-DIMENSIONAL VISION

(71) Applicant: REAL VISION S.R.L., Milano (IT)

(72) Inventor: Sabino PISANI, Rescaldina (Milano) (IT)

(21) Appl. No.: 15/304,258

(22) PCT Filed: Aug. 5, 2013
(86) PCT No.: PCT/IT2013/000220

§ 371 Date: Oct. 14, 2016

(51) Int. Cl. H04N 13/00; A61B 3/00; H04N 13/04

(52) U.S. Cl. CPC H04N 13/0029; H04N 13/0454; H04N 13/0055; H04N 13/0062; A61B 3/0025; A61B 3/0041

(57) ABSTRACT

A device for conversion of formats (f1) of files (F) in a format (f2) for three-dimension (3D) vision, having a receiving unit to receive a plurality of source images (I1i) source in the file (F) in the format (f1), a processing unit having a checking module configured for checking whether the image (I1) is a double right-left channel image, a first processing module configured to represent a left part of the image in a left image (SI1) and a right image (DI1), an interlacing module configured to create an interlaced image (lint) for the image (I1) in which even pixel columns of the interlaced image (lint) are defined as a function of the left image (SI1) and odd pixel columns of the interlaced image (lint) are defined as a function of the right image (DI1), thereby converting the file (F) in the format (f2) for three-dimension (3D) vision.

### TECHNICAL FIELD

The present invention has as a subject-matter a conversion device for format conversion of files for three-dimensional vision.

The present invention has also for subject-matter a method, in particular a computer implemented method, for file format conversion for three-dimensional vision.

The invention, permitting a three-dimensional vision, is applied to medical, scientific and professional areas for production and display of images, short films both stand-alone and in streaming, through wi-fi, for diagnosis, therapy, reporting, industrial graphical displays of mechanical parts and to test the virtual reality, and the following example refers to one of these application fields just to simplify the explanation.

### PRIOR ART

It is well-known that, in ophthalmology to asses the ability (or inability) of a patient eyes, he/her is provided with proper glasses stimulating a stereoscopic vision, thanks to which the brain processes and perceives a three-dimensional (3D) view of a target-object, actually two-dimensionally displayed on a physical support.

The three-dimensional “interpretation” is, therefore just possible through suitably implemented glasses.

However, it is well-known that that in the medical area, in particular in every application in which body portions are subjected to diagnostic methods of different kinds, as for instance echography, tomography and the like, the exam of the resulting images, which is in charge on the experience of the doctor interpreting the images, feels the obvious limits due to the intrinsic poor quality of the images of the two-dimension vision. For these applications, even do not exist fittings, as for instance the above mentioned three-dimension glasses, allowing to overcome, at least partially the actually limited two-dimension vision implemented by the well known diagnostic devices.

The nowadays used known methods for three-dimension vision have then some remarkable drawbacks.

First, if the three-dimension interpretation is already partially possible, they ask for the use of stereoscopic glasses, to be worn by the patient, often causing troubles and disquiet in particularly sensitive subjects.

Second, if the three-dimension interpretation is not yet embodied, they ask the operator, scanning the images, to make a mind effort to interpret in three dimensions two-dimension images.

In both cases it appears the need of an image processing technic allowing an easy and reliable interpretation of two-dimensional images independent from secondary instruments and subjective interpretations, in other words, an automated technic independent from the kind of the input images.

It is an object of the present invention to implement a device and a file format conversion method for a three-dimension vision, which overcomes the above mentioned drawbacks.

It is a specific object of the present invention to implement a device and a file format conversion method for a three-dimension vision, which is of fast execution and accurate in the performing the results.

A further object of the present invention is to provide a computer implemented file format conversion method for three-dimension vision making more efficient the performance and the accuracy of the results.

Another object of the present invention is to embody a device and a conversion method of file formats for three-dimension vision assuring the interoperability with most part of the systems, particularly existing diagnostic systems.

A further object of the invention is to embody a device and a conversion method of file formats for three-dimension vision allowing also a simultaneous vision of two-dimension files assuring a better user comfort.

Another object of the invention is to embody a device and a conversion method of file formats for three-dimension vision allowing a display of image sequences to yield a 3D video.

### SUMMARY OF THE INVENTION

These and other aims are attained by a file format conversion device for three-dimension vision according to what described in the enclosed claims 1 to 7.

The described aims and other aims are also attained by a file format conversion method according to what described in the enclosed claims 8 to 13.

In a preferred embodiment, the method is actuated by means of a computer, according to what described in the enclosed claim 14.

The described aims and other aims are attained by a computer program according to claim 15.

The invention, as described, attains the following technical effects thanks to the non use of glasses for 3D display:
it is natural and less tiring; in fact, as there is not before the eyes a colored polarized sheet, the eye does not expand the pupil (effect due to reduced transmittance and brightness) and consequently the neurosensory and cortical responses of the vision are environmental;
it is avoided the diameter increase of the pupil (due to polarized filters) which, in condition of maximum sight attention, modifies the focusing and consequently changes the convergence stimulus comprising a shift of the perception and focal plane and inducing a sight overload (stress);
allows higher distance tolerance range on the distance for a perfect vision;
the vision without glasses is also stimulating and then educational for people having problems of 3D seeing, and above all, is an easy and amusing experience;
the vision is good even if the environment brightness is precarious;
more comfort as do not exist glasses before the face, above all for people already wearing glasses;
applicability at any temperature condition not being affected by temperature range and then by misting.

Further, the invention attains the following technical effects due to intrinsic interoperability of the device and the method:
the images produced by most of the diagnostic systems are depicted in three-dimension vision;
the production of three-dimension vision can be effected on request;
the three-dimension image flux can be implemented on any conventional electronic device and not specially dedicated;
the three-dimension image flux can be selectively displayed through any hardware of any size even with many degree bent screens without appearing as “distorted”.
the three-dimension image flux can be selectively and simultaneously displayed with two-dimension images to provide a more immediate and efficient use.

The mentioned technical effects/advantages and other technical effects/advantages will result more detailed from the description, here below done, of an embodiment given as indicating and not limiting way with reference to the enclosed drawings.

### BRIEF DESCRIPTION OF THE DRAWINGS

FIG. 1 is a block diagram of the device of the invention.

FIG. 2 is a detail of a block of FIG. 1.

### DETAILED DESCRIPTION OF PREFERRED EMBODIMENTS OF THE INVENTION

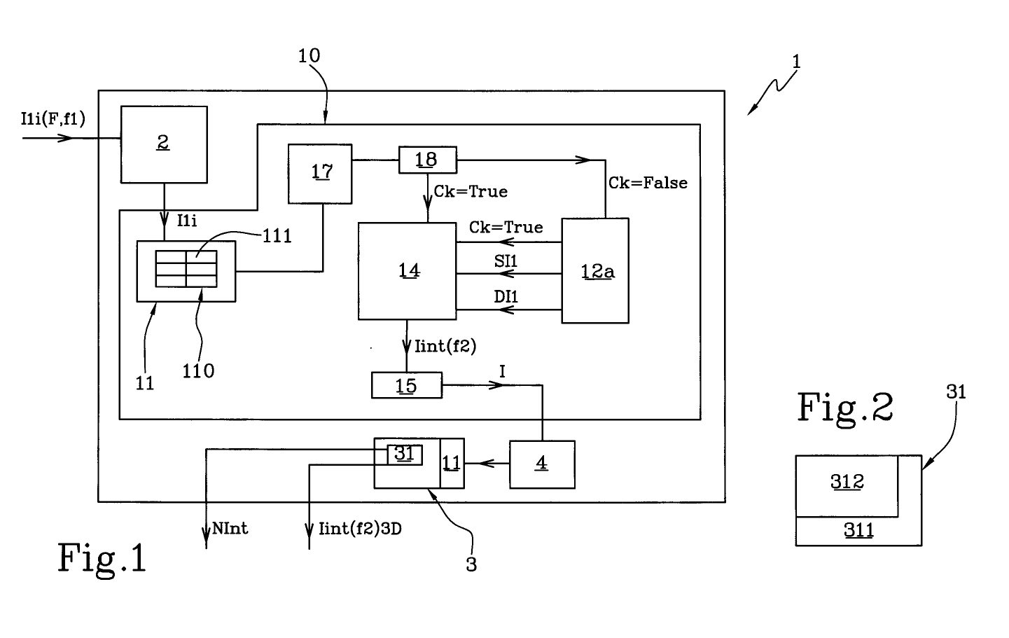

Referring to FIG. 1, it is depicted a device 1 of conversion from first file F formats f1 to a second format f2 for three-dimension vision 3D.

The device 1 comprises a receiving unit 2 configured to receive a plurality of source images I1i in the file F in the first format f1.

Preferably, the first format f1 may be one among JPEG,GIF, PNG, BMP, WEBP, H.263, H.264, AVC, MPEG-4 SP, VP8 or derivatives.

The device in accordance with the invention comprises a processing unit 10. Generally it is to note that in the present context and in the following claims, the processing unit 10 is depicted as subdivided in different functional modules (memory modules or operating modules) to the only purpose to clearly and completely describe the functions.

Actually such a processing unit 10 may consist of just one electronic device, suitably programmed to implement the described functions, and the different modules may correspond to hardware entities and/or software routines forming the programmed device. Alternatively or in addition, said functions may be performed by a plurality of electronic devices on which the above mentioned functional modules may be arranged.

The processing unit 10 may avail itself of one or more processors for the carrying out of the instructions contained in the memory modules.

The above mentioned functional modules may be further distributed on different either local or remote computers in accordance with the architecture of the network in which they reside.

The processing unit 10 comprises a first setting module 11 configured to import the plurality of source images I1i in the first format f1; in a first embodiment of the invention, the plurality of source images I1i is representative of outputs of diagnostic systems, as for instance, instruments for ultrasound echography, tomography and the like. The first setting module 11 is further configured to prepare a representation area 110 allowing a representation of the source image I1.

Preferably, the representation area 110 is changing in accordance with the acquisition of the source images I1i.

Preferably, the representation area 110 is a bitmap.

Preferably, the image is depicted through canvas.

The bitmap is manageable through a scripting language in which the input is an image drawing with canvas graphical instruments or an image imported in a first format f1.

As it is well known, the canvas consists of a drawable area, defined in HTML code with the height and width attributes. The code JavaScript can accede the area with a complete function set for the drawing, similar to those common for other API 2D, so allowing the dynamic generation of drawings.

The processing unit 10 according to the invention, includes a checking module 17, active on every image I1 of source image plurality I1i.

The checking module 17 is configured to check whether the image I1 in the file F is a double right-left channel image.

In fact, such an image could be a single 2D image which could be split in two for the here below described functions.

The check module 17 is configured to write a check worth CK in a memory module 18 as a function of the effected check; in other words, the checking module 17 checks that the image I1 is a double right-left channel image and stores the outcome of such a check to discriminate among the following processes which will be effected on the image I1.

The processing unit 10 comprises a first processing module 12a.

The first processing module 12a is configured to be actuated as a function of the check worth CK=FALSE to represent the image I1 in double right/left channel format.

In other words, if the input image I1 is not in the required format to be then converted in a format for 3D display, the image is prepared in the configuration suited for such a display.

In the case that the image is not in the required format, that is CK=FALSE, the first processing module 12a is set to represent a left part of the image I1 as a left image SI1 and the right part of the image I1 as a right image DI1.

In particular, the first processing module 12a is configured to depict the left part of the image I1 as a left image SI1 with half width with respect to the image I1, and the right part of the image I1 as a right image DI1 with half width with respect to the image I1.

In accordance with the invention, the processing module 12a is further configured to set up the check worth CK=TRUE at the end of the above mentioned representation.

Advantageously, according to the invention the processing unit 10 comprises an interlacing module 14 configured to be activated as a function of the check worth CK=TRUE.

In such a case, the interlacing module 14 is configured to create an interlaced lint image for the image I1.

Particularly, even pixel columns of the interlaced lint image are defined as a function of the left image SI1 and odd pixel columns of the interlaced lint image are defined as a function of the right image DI1.

The interlacing allows a better transmission quality without increasing the band width.

Through such a processing, is embodied a file F conversion in the second format f2 for three-dimension 3D vision of the interlaced lint image.

According to the invention, the device 1 comprises a display unit 3 configured to display at least the interlaced (lint) image in three-dimension vision in the second format f2.

The processing unit 10 comprises an activation module 15 configured for an activation T of a three-dimension display of the interlaced lint image.

According to the invention, the device 1 comprises a parallax activation unit 4, associated with the display unit 3.

Advantageously, according to the invention, the parallax activation unit 4 is configured to actuate a parallax barrier 41 in function of an activation T received by the activation module 15 for a display on the display unit 3.

According to the invention, the processing in sequence of the source image I1i determines a video 3D effect on the display unit 3.

Such a 3D video display assures an excellent play and vision quality to the user starting from simple images in a first format f1.

Advantageously, according to the invention, the display unit 3 is configured to display not interlaced images Nlint and interlaced images lint as a function of modifications to the imaging area 110 due to the acquisition of the source images I1i.

To this purpose, the display unit 3 comprises a user interface 31, and the unit 3 is configured to display the not interlaced images Nlint in at least a first portion 311 of the user interface 31, and the interlaced images lint in at least a second portion 312 of the user interface 31.

In other words, on the display of the device, at least an area will be dedicated to the 3D display, preferably of the discussed image, and at least another area will be dedicated to comprise selection push-buttons which do not require any 3D display.

In other words, the representation area 110 is configured to be represented on different layers 111 in function of a predefined 3D or 2D imaging.

Otherwise told, the layers 111 are set in order to be allocated to interlaced lint or not interlaced Nlint images.

The representations will be displayed on displayable areas, for instance the first and the second portion 311 and 312.

To summarize, the imaging area 110 comprises a plurality of layers 111 defined in function of predefined allocated 3D or 2D imaging.

Advantageously, according to the invention the parallax activation unit 4 is set to actuate a parallax barrier 41 selectively in function of the considered layer 111.

The obtained technical effect is the predisposition of a displayable area, for instance the first and second portion 311 and 312, on the display device 3 selectively allocated to 3D or 2D display with obvious advantages of selective and optimized processing of the images/image sequences to be shown.

The invention describes also a conversion method of first file F formats f1 in a second format f2 for three-dimension 3D vision.

In accordance with the invention, the method comprises the steps for receiving a plurality of source images I1i in the file F in a first format f1.

The method according to the invention provides to prepare an representation area, preferably a bitmap 110, allowing a representation of the source image I1.

The method further comprises the step for checking in every source image I1 of the plurality of the source images I1i, whether the source image I1 is a double right-left channel source image.

Further, the method provides to write a check worth CK in a memory module 18 as a function of the check; in other words, if the image is a double right-left channel image, then the method provides to write a worth CK=TRUE, otherwise CK=FALSE.

The method further comprises the step to represent the source image I1 in a double right-left channel format if the check worth is CK=FALSE.

Consequently, a further step is representing the left part of the source image I1 in a left image SI1 and the right part of the image in a right image DI1, so that the image is prepared for a three-dimension vision.

Once finished the representation, the method provides the creation of an interlaced image.

Precisely, the method provides, as a function of the check worth CK=TRUE, to create an interlaced image lint for the source image I1 wherein the even pixel columns of the interlaced image lint are defined as a function of the left image SI1 and the odd pixel columns of the interlaced image lint are defined as a function of the right image DI1; such an operation determines a conversion of the file F in the second format f2 for three-dimension 3D vision.

At last, the method provides to activate a three-dimension display of the interlaced image lint.

In particular, advantageously, according to the invention the method provides to activate a parallax barrier 41 as a function of the activation of the three-dimension display of the interlaced image lint, and to display at least the interlaced image lint in three-dimension display in the second format f2.

According to the invention, the processing of the source image I1i in sequence determines a video 3D effect on the display unit 3.

Such a 3D video display assures an excellent display and vision quality to the user starting from simple images in a first format f1

Advantageously, according to the invention, the method provides to display not interlaced images Nlint and interlaced images lint as a function of modifications to the representation area 110 dictated by the acquisition of the source images I1i.

In particular, the method provides to display the not interlaced images Nlint in at least a first portion 311 of a user interface 31 and display the interfaced images lint in at least a second portion 312 of the user interface 31, as depicted in FIG. 2.

In other words, the method provides represent in 3D or 2D on different layers 111 of the representation area 110.

Otherwise stated, the layers 111 are set to be allocated to image representations lint or Nlint.

The representations will be then displayed on displayable areas, for instance the first and second portion 311 and 312.

The method further provides to activate the parallax barrier 41 selectively as a function of the considered layer 111.

In short, the conversion method provides to preset a plurality of layers 111 in the representation area 110 and to allocate a 3D or 2D representation as a function of the defined presetting.

The obtained technical effect is the presetting of a displayable area, for instance the first and second portion 311 and 312, on the display device 3 selectively allocated to 3D or 2D representation with evident advantages of selective and optimized processing of the images/image sequences to be shown.

In a preferred embodiment of the invention, the conversion method is characterized in that it is a computer implemented conversion method.

In a preferred embodiment, a computer program comprises instructions which, when executed, allow to carry out one or more of the described method steps.

1. A conversion device of first format (f1) of a file (F) into a second format (f2) for three-dimension (3D) vision, wherein the device comprises:
a receiving unit configured to receive a plurality of source images (I1i) in said file (F) in said first format (f1);
a processing unit comprising:
a checking module, acting on every source image of said plurality of source images (I1i), configured to:
check whether said source image in said file (F) is a double right-left channel image;
write a check worth (CK) in a memory module as a function of said check;
a first processing module configured to:
start in function of said check worth (CK)=FALSE to represent said source image in a format of double right and left channel;
represent a left part of said source image in a left image (SI1) and a right part of said source image in a right image (DM);
set said check worth (CK)=TRUE;
an interlacing module configured to:
start in function of said check worth (CK)=TRUE;
create an interlaced image (lint) for said source image wherein even pixel columns of said interlaced image (lint) are defined as a function of said left image (SI1) and odd pixel columns of said interlaced image (lint) are defined as a function of said right image (DM), thereby converting said file (F) in said second format (f2) for three-dimension (3D) vision of said interlaced image (lint); and
an activation module configured for an activation (T) of a three-dimension display of said interlaced image (lint).

2. The conversion device according to claim 1 wherein said processing unit comprises a first setting module configured for:
importing said plurality of source image (I1i) in said first format (f1), wherein said source image plurality (Hi) is preferably representative of diagnostic systems output; and
preparing a representation area configured to allow a representation of said source image.

3. The conversion device according to claim 1 further comprising:
a display unit configured to display at least said interlaced image (lint) in three-dimension vision in said second format (f2);
a parallax activation unit, associated to said display unit, configured to activate a parallax barrier as a function of said activation (T) received from said activation module for a display on said display unit.

4. The conversion device according to claim 3 wherein said display unit is configured to display not interlaced images (Nlint) and said interlaced images (lint) as a function of modifications to said representation area dictated by the acquisition of said plurality of source images (I1i).

5. The conversion device according to claim 4 wherein said display unit comprises a user interface, and said display unit is configured to display said not interlaced images (Nlint) in at least a first portion of said user interface, and said interlaced images (lint) in at least a second portion of user interface.

6. The conversion device according to claim 2 wherein said representation area comprises a plurality of layers defined as a function of a predefined allocated 3D or 2D representation.

7. The conversion device according to claim 1 wherein said first processing module is configured to represent said left part of said source image in said left image (SI1) with half width with respect to said image, and said right part of said source image in said right image (DM) with half width with respect to said source image.

8. A conversion model of first formats (f1) of files (F) in a second format (f2) for three-dimension (3D) vision comprising:
receiving a plurality of source images (I1i) in said file (F) in said first format (f1);
checking in every source image of said plurality of source images, whether said source image in said file (F) is a double right-left channel image;
writing a check worth (CK) in a memory module as a function of said check;
representing said source image in a double right and left channel format if said check worth (CK)=FALSE;
representing said left part of said source image in a left image (SI1) and said right part of said source image in a right image (DI1);
setting said check worth (CK)=TRUE;
as a function of said check worth (CK)=TRUE, creating an interlaced image (lint) for said source image wherein even pixel columns of said interlaced image (lint) are defined as a function of said left image (SI1) and odd pixel columns of said interlaced image (lint) are defined as a function of said right image (DI1), thereby converting said file (F) in said second format (f2) for three-dimension (3D) vision of said interlaced image (lint); and
activating a three-dimension display of said interlaced image (lint).

9. The conversion method according to claim 8 further comprising the steps of:
setting said plurality of source images (I1i) in said first format (f1), wherein said plurality of source images (I1i) is preferably representative of diagnostic system outputs; and
preparing a representation area configured to allow a representation of said source image.

10. The conversion method according to claim 8 further comprising:
displaying at least said interlaced image (lint) in three-dimension vision in said second format (f2);
activating a parallax barrier in function of said start for a three-dimension display of said interlaced image (lint).

11. The conversion method according to claim 8 comprising:
displaying not interlaced images (Nlint) and said interlaced images (lint) as a function of modifications to said representation area dictated by the acquisition of said source images.

12. The conversion method according to claim 11 comprising:
displaying said not interlaced images (Nlint) in at least a first portion of a user interface; and
displaying said interlaced images (lint) in at least a second portion of said user interface.

13. The conversion method according to claim 9 comprising:
presetting a plurality of layers in said representation area; and
allocating a 3D or 2D representation as a function of said defined presetting.

14. The conversion method according to claim 8 wherein it is a computer implemented method.

15. A computer program comprising instructions which, when carried out, allow to implement the method according to claim 8.
